(19) United States
(12) Patent Application Publication
(10) Pub. No.: US 2025/0281088 A1
(43) Pub. Date: Sep. 11, 2025

(54) TRAINING, CONFIGURING, APPLYING, AND ITERATIVELY IMPROVING AI-LANGUAGE-MODEL-BASED HAPPINESS AND WELLBEING SUPPORT SYSTEMS

(71) Applicant: Matter Neuroscience Inc., Boulder, CO (US)

(72) Inventor: Axel BOUCHON, Kleinmachnow (DE)

(73) Assignee: Matter Neuroscience Inc., Boulder, CO (US)

(21) Appl. No.: 19/070,879

(22) Filed: Mar. 5, 2025

Related U.S. Application Data
(60) Provisional application No. 63/563,100, filed on Mar. 8, 2024.

(51) Int. Cl. A61B 5/16; A61B 5/00; A61B 5/055

(52) U.S. Cl. CPC A61B 5/165; A61B 5/0042; A61B 5/055; A61B 5/4836; A61B 5/486; A61B 5/7267; A61B 2576/026

(57) ABSTRACT

Disclosed herein are systems and methods for training an AI language model for assessing and improving happiness and wellbeing of a human user, the method comprising receiving a pre-trained AI language model; receiving a first training data set comprising non-user-specific training data comprising brain imaging data; applying one or more supervised training protocols based on the first training data set to modify the pre-trained AI language model to generate a non-user-specific language model configured for happiness and wellbeing support of human users; receiving a second training data set comprising user-specific training data comprising brain-imaging data for a specific user; and applying one or more supervised training protocols based on the second training data set to modify the non-user-specific language model to generate a user-specific language model configured for happiness and wellbeing support of the specific user.

### CROSS-REFERENCE TO RELATED APPLICATIONS

This application claims priority to and the benefit of U.S. Provisional Application No. 63/563,100, filed Mar. 8, 2024, the contents of which are incorporated herein in its entirety.

### BACKGROUND

Large language models (LLMs), such as OpenAI's ChatGPT series, Gemini, and Mistral's open-source models, have been built based on large databases comprising various sources of human communication. The primary objective of those models is to provide a humanoid bot that is capable of fluent speech and communication, such that the bot does not fall short in language comprehension or language generation speed during interaction with a normal human user. Given ease of access, data corpuses available via the internet have been leveraged in training and otherwise configuring existing LLMs for the purpose of general-purpose language interaction with human users.

Due to the use of publicly-available internet content for training, reliable correctness and truth in responses generated by existing LLMs has not been achieved. In particular, existing LLMs have significant inherent deficits in their performance in areas of scientific data and scientific knowledge, especially in scientific areas that are emerging or are facing active expert debate. One such area of emerging scientific knowledge and active expert debate is the area of human wellbeing and human happiness. Because of the inherent limitations due to the manner in which existing LLMs are trained, particularly in that their training data does not include deep and emerging scientific information required for effective wellbeing and happiness support, they are thus not equipped to effectively support user needs in these areas of human wellbeing and happiness. Also, the underlying learning structure is often missing a set of rules and a knowledge base that the model can rest and build off from (e.g., an inference engine).

Furthermore, due to the use of publicly-available internet content for training, existing LLMs are not equipped to effectively provide personalized support in the manner that is necessary for effective wellbeing and happiness support for individual human users. While LLMs are capable of interaction in a manner that might linguistically approximate advice regarding wellbeing and happiness, their capabilities do not allow them to weigh personal user preferences over the averaging base of the internet-sourced data corpuses based on millions of people and trillions of posts.

Further still, due to the manner in which existing LLMs are trained, configured, and deployed, they are systematically blind to changing behavioral and emotional needs in a person, at least because emotional experiences are incompletely captured by the written-language training data used to generate existing LLMs. And, even during deployment in which existing LLMs interact with individual users, information gleaned regarding the individual user is limited to text-based information that cannot fully capture the user's emotional experience, making it impossible for the LLM to effectively learn personal emotional and wellbeing needs of the user.

Accordingly, there is a need for LLM-based (or other language-model based) systems that are trained, configured, and deployed in a manner that allow the LLM to access and leverage deep and emerging scientific information regarding human happiness and wellbeing, and that allow the LLM to access and leverage personalized information about individual human users in order to provide effective and adaptive individualized happiness and wellbeing support.

### SUMMARY OF THE INVENTION

Disclosed herein are systems and methods for training, configuring, applying, and iteratively improving AI-language-model-based happiness and wellbeing support systems. The methods and systems include two supervised training processes are applied to respectively train a non-user-specific LLM-based happiness intelligence system and a personalized, user-specific LLM-based happiness intelligence system. Following the two supervised training processes, two continuous training methods may be applied to improve understanding by the intelligence system(s) of the emotional needs of the human species generally and of a single human user.

In some embodiments, systems, methods, and non-transitory computer readable storage media are provided herein for training an AI language model for assessing and improving happiness and wellbeing of a human user, the system comprising one or more processors and memory storing instructions configured to be executed by the one or more processors to cause the system to: receive a pre-trained AI language model; receive a first training data set comprising non-user-specific training data comprising brain imaging data; apply one or more supervised training protocols based on the first training data set to modify the pre-trained AI language model to generate a non-user-specific language model configured for happiness and wellbeing support of human users; receive a second training data set comprising user-specific training data comprising brain-imaging data for a specific user; and apply one or more supervised training protocols based on the second training data set to modify the non-user-specific language model to generate a user-specific language model configured for happiness and wellbeing support of the specific user.

In some embodiments, systems, methods, and non-transitory computer readable storage media are provided herein for using an AI language model to generate a recommendation for an action to induce an improved happiness and wellbeing state of a user, the system comprising one or more processors and memory storing instructions configured to be executed by the one or more processors to cause the system to: receive input indicating a current happiness and wellbeing state of the specific user; generate, based on the input indicating the current state, an indication of an unmet happiness or wellbeing need of the user; and apply a user-specific language model to generate, based on the indication of the unmet need of the user, a first output comprising a first recommendation for an action to induce an improved happiness and wellbeing state of the user, wherein the user-specific language model is trained based brain-imaging data for the user.

### BRIEF DESCRIPTION OF THE FIGURES

FIGS. 1A-1B illustrates a diagram of a continuous training model for large language models (LLMs) including a personal happiness intelligence (PHI) training model and a hive happiness intelligence (HHI) training model, in accordance with some embodiments.

FIG. 2 illustrates a diagram of the hive happiness intelligence (HHI) training model shown in FIGS. 1A-1B, in accordance with some embodiments.

FIG. 3 illustrates a diagram of the personal happiness intelligence (PHI) training model shown in FIGS. 1A-1B, in accordance with some embodiments.

FIG. 4 illustrates a diagram for updating the hive happiness intelligence (HHI) training model shown in FIGS. 1 and 2 with expert data, in accordance with some embodiments.

FIGS. 5A-5B illustrates an exemplary use case of the continuous training model shown in FIGS. 1A-1B, in accordance with some embodiments.

FIG. 6 illustrates a computer for executing the processes disclosed herein, in accordance with some embodiments.

### DETAILED DESCRIPTION

Disclosed herein are systems and methods for training, configuring, applying, and iteratively improving AI-language-model-based happiness and wellbeing support systems. While the disclosure herein may refer to LLMs, the techniques described herein may be applied to other AI-based language models, as would be understood by a person of skill in the art. In some embodiments, two supervised training processes are applied to respectively train a non-user-specific LLM-based happiness intelligence system and a personalized, user-specific LLM-based happiness intelligence system. Following the two supervised training processes, two continuous training methods may be applied to improve understanding by the system (including one or both of the LLM-based systems) of the emotional needs of the human species generally and of a single human user.

As noted above, existing LLMs have been trained and otherwise developed primarily using publicly-available internet content. Publicly available data often does not include deep and emerging scientific information that is required for effectively supporting user needs in the areas of human wellbeing and happiness. Accordingly, existing LLMs are unable to effectively provide personalized happiness and wellbeing support for individual users. Moreover, existing LLMs are blind to the everchanging emotional and behavioral needs of a human and thus are unable to completely learn from the person's emotional experience to support the emotional wellbeing of the person. The disclosed systems and methods address the above-identified needs by providing a personalized happiness and wellbeing intelligence system that is continuously refined based on learnings of the user and non-user-specific learnings (e.g., data-backed findings, learnings of other users, etc.).

In some embodiments, in a first training step, a large training data set including essential and/or peer-reviewed literature in philosophy, psychology, behavioral science, molecular neuroscience, and brain imaging is provided. The training data set is reviewed and optionally filtered, and is weighted for importance and correctness, e.g., by human subject-matter experts, by one or more automated analysis algorithms, or both. This weighted database of training data may be referred to as a general human wellbeing database. This human wellbeing database may be used to train (e.g., using supervised training) an LLM for correctness and truth on the topic of human wellbeing and happiness. In some embodiments, training the LLM may comprise applying additional training steps (e.g., supervised training) to a pre-trained LLM. For example, an LLM trained initially using a corpus of general-purpose internet-sourced training data may then be subsequently trained further using weighted data from the general human wellbeing database. This training process may generate an improved and wellbeing-focused LLM.

Following the first training step explained above, a second training step leveraging personal user data may be applied. In some embodiments, application of the second training step leveraging personal user data may transform the LLM-based system from a general-purpose wellbeing-optimized AI system into a personalized wellbeing-optimized system specifically configured for use by a specific human user. In some embodiments, training data used in the second training step may include clinical physiology data, clinical biochemistry data, data from full genome analysis, and/or data from full brain analysis using structural and functional MRI in 7 Tesla (or higher) brain scanners. In some embodiments, training data used in the second training step may include behavioral data covering one or more self-assessment questionnaires, peak memory data, and/or actual memory data collected using the Matter Protocol (e.g., as explained in PCT publication WO 2024/026437 A1, incorporated herein by reference). During and/or after the second training step, effectiveness of the trained system in providing personal wellbeing recommendations may be tested, iterated, and improved under supervision of experts and/or the target end-user for whom the system is being configured.

Following the second training step explained above, a third training step may be applied to leverage information based on the target human user's activities and the user's associated emotional feedback. This third training step may be a continuous training step that gleans and leverages information regarding unmet emotional needs of the user.

On the basis of the collected personal data, an assessment module may address the behavioral and emotional status of the person using various emotion assessments. The assessment module outputs may then serve as the input for a need module that may determine one or more actual unmet needs of the user. The need module then connects the brain molecule calculations to distinct needs of the user, for example by applying a matrix that connects unmet needs and neurotransmitters. A recommendation module may provide a personalized recommendation to fill the one or more determined unmet needs. Recommendations generated by the user-specific and/or non-user-specific models may then be assessed by a decision module that may enable a user to execute a manual process in which the user provides an input indicating which of a plurality of recommendations from one or both of the models is preferred for execution.

Subsequent actions taken by the user following the output provided by the recommendation may be manually recorded and/or automatically sensed by one or more sensors, and may (in combination with additional manual input, additional biodata monitoring, and/or additional data regarding location, time, colocation or communication with influencing people, etc.) provide a positive or negative reinforcement loop for the system to learn continuously.

Following the third training step explained above, a fourth training step may be applied to ensure that information regarding decisions that increase a single user's wellbeing and happiness is fed back to the improved and wellbeing-focused non-user-specific LLM. The fourth training step may be a continuous training step that includes extracting non-personal data from a specific user's data and delivering it back to the non-user-specific LLM model.

Continued improvement of the non-user-specific LLM model may be achieved on an intermittent or continuous basis by providing additional peer-reviewed expert publications addressing the topic of wellbeing and happiness in the areas of philosophy, psychology, behavioral science, molecular science, and/or brain imaging and diagnostics. This may include iteratively updating (and/or re-performing) the first training step described above, and/or may include performing a continuous training/refinement step similar to the fourth training step explained above.

As used herein, the term “outcome” may refer to any one or more of: the user's selection of a recommendation, the user's action or non-action following a recommendation, and/or the user's emotional, neurological, psychological, and/or biophysical state following performance or non-performance of a selected action.

Unless explicitly stated otherwise herein, it is to be understood that the personalized happiness and wellbeing support system and associated methods may be constructed from a rules-based inference engine, large reasoning model, etc. rather than a large learning model or AI-based approach.

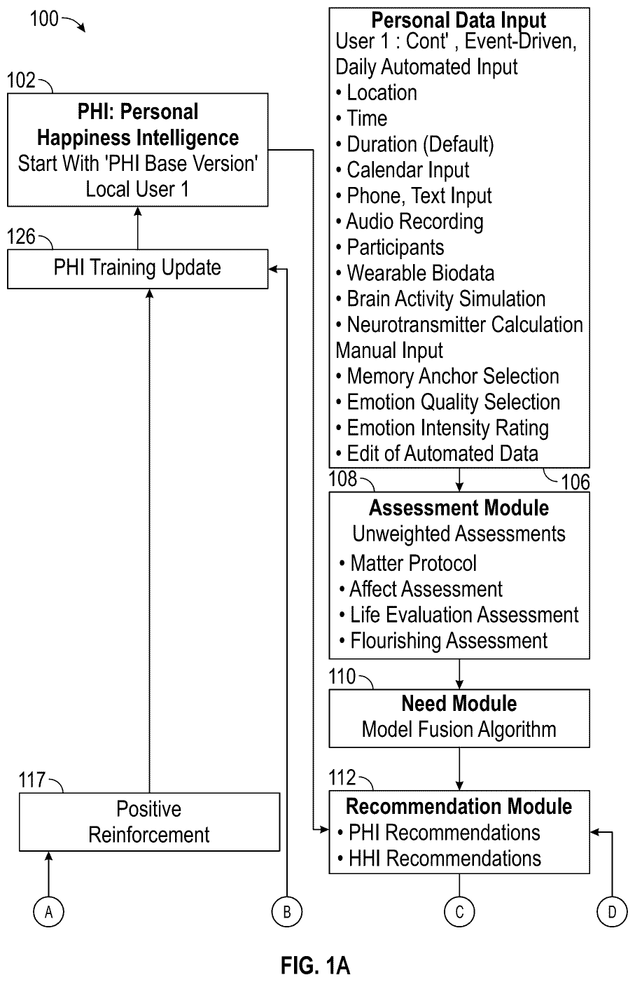

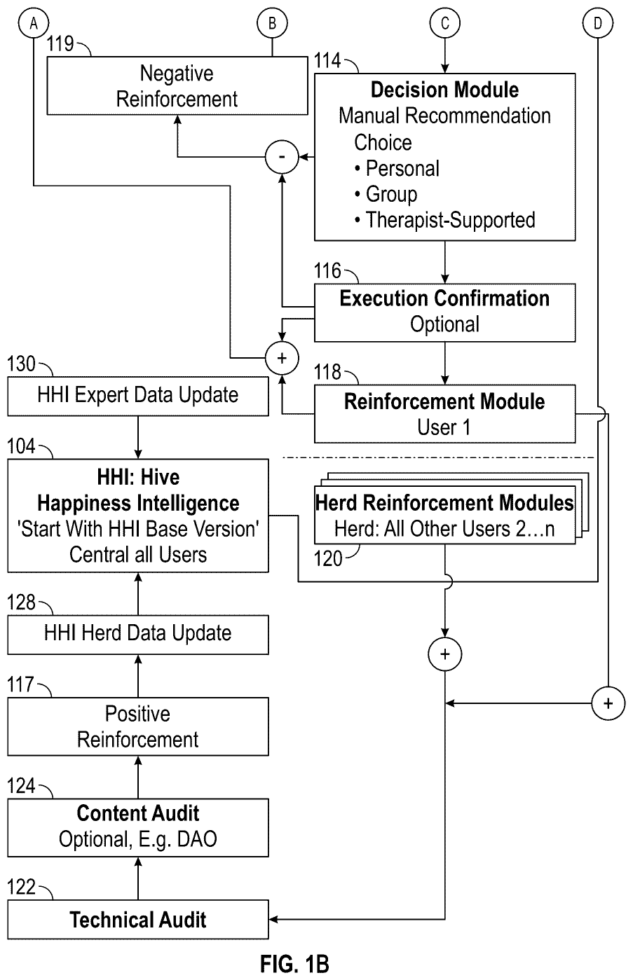

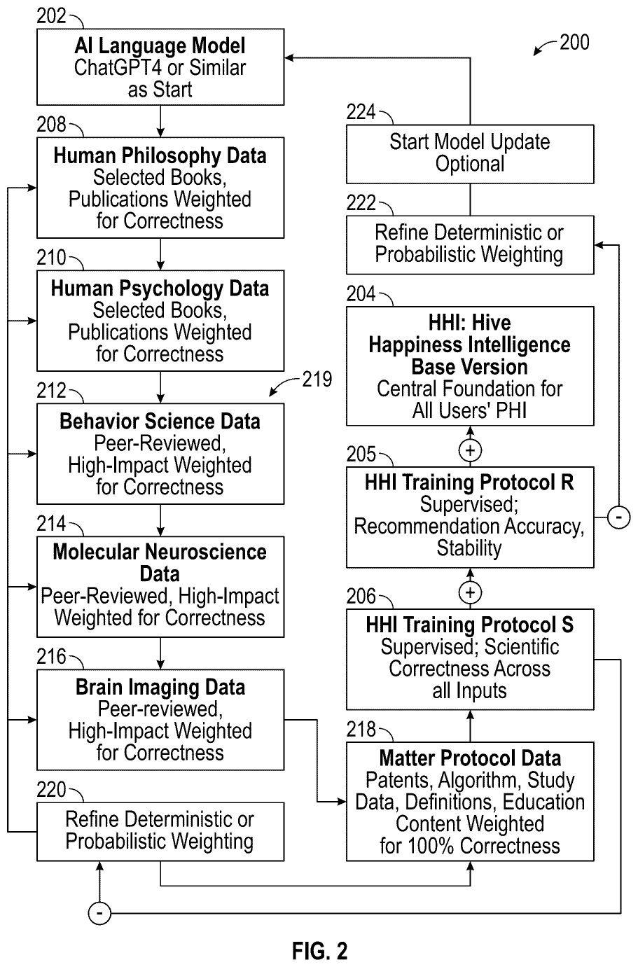

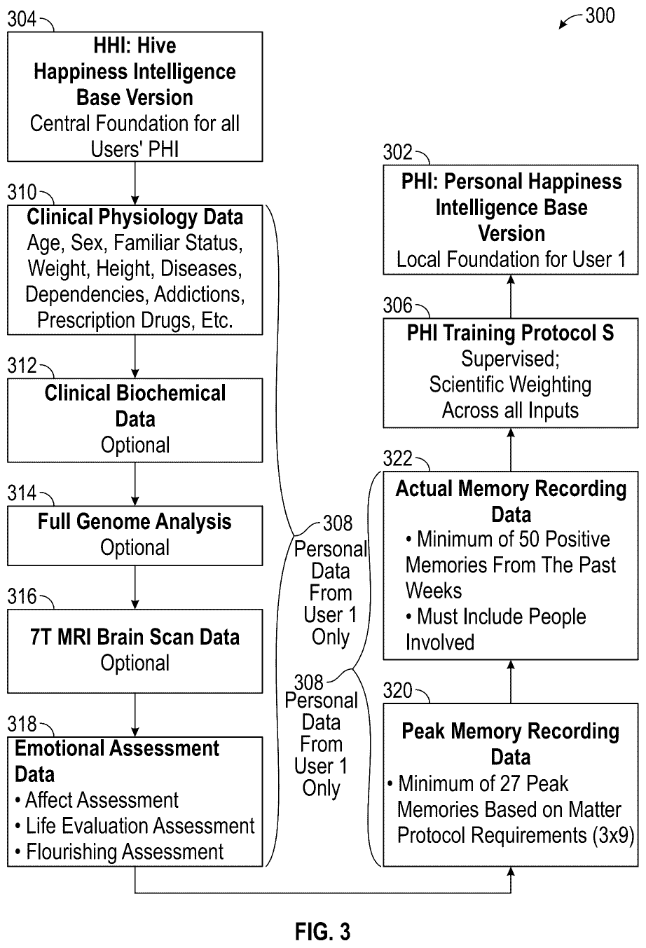

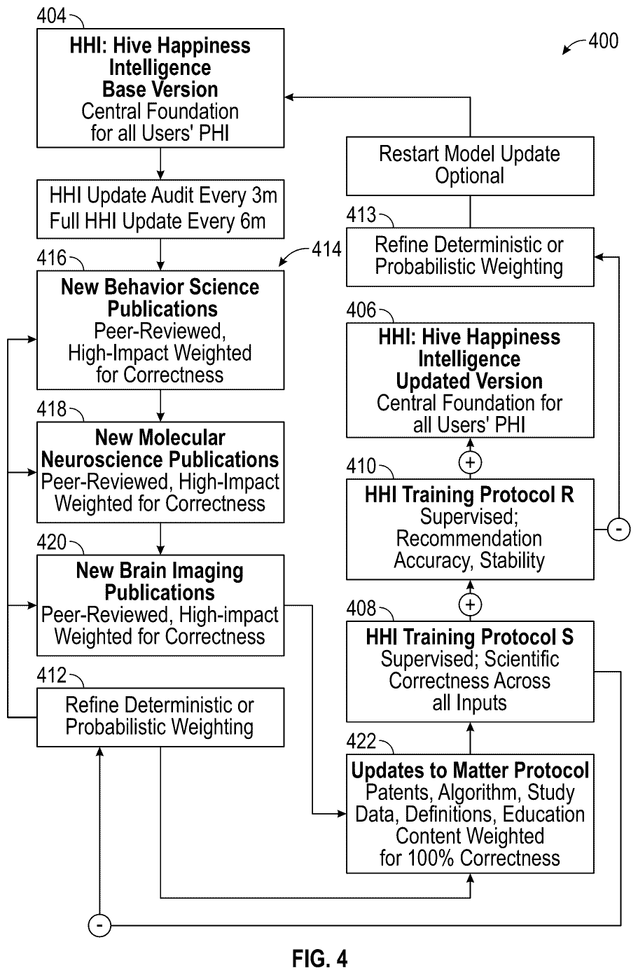

FIGS. 1A-1B depicts a method 100 for application and continuous training/refinement of a personalized LLM-based happiness and wellbeing support system 102 (referred to in the figures as personal happiness intelligence, or PHI) and a non-user-specific LLM-based happiness and wellbeing support system 104 (referred to in the figures as hive happiness intelligence, or HHI). Training of the personalized system 102 is described in greater detail with respect to FIG. 3. Training of the non-user-specific system 104 is described in greater detail with respect to FIGS. 2, 4.

As shown in FIGS. 1A-1B, both the personalized system 102 and the non-user-specific system 104 may be used to generate recommendations based on user needs. Specifically, personal user data 106 may be subject to various assessment protocols by an assessment module 108, and a need module 110 may determine, based on output from the assessment module 108, various psychological and/or neurological needs of the user. Each of the modules described herein may be embodied by one or more processors and programs configured to execute on one or more processors. The chain of modules beginning with the personal data input 106 and ending with the optional execution confirmation 116 may otherwise be referred to as a third training step of the AI language model for assessing and improving happiness and wellbeing of a human user.

The personal user data 106 may be automatically collected, for example from the user's phone or other electronic device. Collected data may include user location, time, duration, use of certain apps (e.g., phone, text), and/or use of certain media. Collected data may include biodata provided by one or more physiological sensors, for example as provided in a fitness tracker; the biodata may include, e.g., heart-rate variability, skin conductivity, body temperature, etc. In some embodiments, collected data such as biodata may include data received and/or data collected from one or more sensors configured to measure one or more parameters including, but not limited to cardiac interbeat interval (IBI), cardiac pre-ejection period (PEP), number of skin conductance responses (SCRs), respiratory sinus arrhythmia (RSA), and/or mean arterial pressure (MAP). Each of these is described as follows.

Cardiac Interbeat Interval (IBI) is measured (in ms), for example, by sensors based on (1) electrical activity (such as Electrocardiogram based on wet electrodes, dry electrodes, or capacitive electrodes), (2) sensors detecting arterial pulse using photoplethysmography (PPG), or sensors such as PhysioCam (PhyC), a non-contact system capable of measuring arterial pulse with sufficient precision to derive HRV during different challenges, (3) sensors based on mechanical activity (balistocardiogram (BCG) using e.g. Hydraulic sensors, EMFi film sensors, Accelerometer), radio frequency or seismocardiogram (SCG) using e.g. Accelerometer, Laser Doppler Vibrometer, Laser Speckle Vibrometry, Airborne Ultrasound or gyrocardiogram (GCG) using gyroscope or Laser speckle vibrometer, and/or (4) Forcecardiography.

Cardiac Pre-Ejection Period (PEP) is measured (in ms), for example, by one or more of the same or similar sensor types as described above with reference to IBI (optionally, with a preference for Forcecardiography and/or Seismocardiography). PEP may be measured by simultaneously collecting both ECG, as described earlier, and impedance cardiography.

Skin Conductance Responses (SCRs) is measured (by number of valid SCRs), for example, by sensors detecting galvanic skin response such as Ag/AgCl, stainless steel, silver, brass, and gold electrodes, Flexcomp Infiniti physiological monitoring and data acquisition unit, Empatica E4 and Refa System, Microsoft Band 2, Empatica E4, Health Sensor Platform, BITalino, Polar H6, Wearable Zephyr BioHarness 3, and/or Obimon EDA.

Respiratory Sinus Arrhythmia (RSA) is measured (in ms²), for example, by electrocardiogram sensors such as any one or more of those described above.

Mean Arterial Pressure (MAP) is measured (in mmHg), for example, by (1) pressure-based methods (e.g. vascular unloading technique, arterial tonometry), (2) ultrasound-based methods, and/or (3) deep-learning based methods using data from PPG or ECG.

Collected data, optionally including biodata, may be augmented and/or annotated using emotion data for the user, which may include manually entered data provided by the user indicating distinct emotions (e.g., event-based emotion data collected via the Matter protocol, for example as explained in U.S. Pat. No. 12,207,927, and in PCT Patent Application PCT/US2023/071174, each of which are incorporated herein by reference). Collected data, optionally including biodata, may be augmented and/or annotated using emotion data for the user, which may include wellbeing assessment data (e.g., Life evaluation assessment, Affect assessment, Flourishing assessment, Matter Protocol assessment, etc.).

Using the collected personal data 106, the assessment module 108 may address the behavioral and emotional status of the person using various emotion assessments, such as the Matter protocol (with or without 7T fMRI brain scan confirmation), life evaluation assessment, affect assessment, and/or Flourishing-self-assessment (as described in PCT Patent Application PCT/US2023/071174). In some examples, the outputs may be weighted at assessment module 108 for significance by a weighting function that provides a quantification of associated brain molecules (e.g. neurotransmitters), brain growth factors, and/or memory formation biomarkers, as the output of the assessment module 108. The assessment module 108 outputs may then serve as the input for the need module 110 that may determine one or more actual unmet needs of the user. The need module 110 can connect the brain molecule calculations to distinct needs of the user, for example by applying a matrix that connects unmet needs and neurotransmitters, including optionally by weighing distinct input factors from the assessments. Such a connecting matrix may allow the system to identify key unmet needs for the user, and may translate the top (e.g., top three) unmet needs in an LLM search prompt as an output.

The determined psychological and/or neurological needs of the user may then be processed, alone and/or in combination with the underlying data used for said determinations, by a recommendation module 112. In some examples, the generated top unmet need LLM prompts (described above) may serve as inputs for the recommendation module 112 that use the prompts as inputs for processing by the user-specific 102 and/or non-user-specific models 104.

The recommendation module 112 may leverage one or both of the personalized system 102 and the non-user-specific system 104 to generate recommendations for a specific user, based on the output of the need module 110, to meet the user's psychological and/or neurological needs and to thereby improve the user's happiness and wellbeing. For example, the user-specific system 102 can have knowledge of user-specific learnings of the preferences and behaviors of the user, and further can have knowledge of the link between the specific user's emotions and neurotransmitters (as discussed above, the positive emotion-to-neurotransmitter matrix). On the other hand, the non-user-specific system 104 can have knowledge of the biological benefits from literature, Internet data, etc. The recommendation module 112 can reconcile these learnings from the user-specific and non-user-specific systems 102, 104 to generate recommendations for the specific user.

Recommendations generated by the user-specific 102 and/or non-user-specific models 104 may then be assessed by a decision module 114 that may enable a user to execute a manual process in which the user provides an input indicating which of a plurality of recommendations from one or both of the models is preferred for execution (e.g., illustrated as execution confirmation 116 in FIGS. 1A-1B).

The decision made by the user for each identified area of unmet need (e.g., positive for one, negative for 19 or more) may provide an immediate feedback loop to one or both of the models 102, 104 to reinforce future recommendation choices. In some embodiments, decisions made by the user may be used unto themselves for feedback and continuous training of the one or more models 102, 104. In some embodiments, the one or more user decisions may be used in combination with information about an emotional, neurological, psychological, and/or biophysical state following performance (or non-performance) of the action selected by the user.

As further shown in FIGS. 1A-1B, the personalized system 102 and the non-user-specific system 104 may both be integrated into a reinforcement learning process 118 by which recommendations that are selected (e.g., via a decision module 114) and/or actions that are (or are not) taken on the basis of recommendations made by the recommendation module 112 (illustrated in FIGS. 1A-1B by the execution confirmation module 116) may be used to provide positive and/or negative reinforcement 117, 119 to the personalized system 102 and/or to the non-user-specific system 104. The reinforcement modules 118, 120, may otherwise be referred to as a fourth training step of the AI language model for assessing and improving happiness and wellbeing of a human user. The fourth training step may be a continuous training step that includes extracting non-personal data from a specific user's data (e.g., scrubbing personal information from a specific user's data), such that sufficiently depersonalized and/or anonymized data regarding a user's recommendations, actions, and neurological/psychological outcomes are delivered it back to the non-user-specific LLM model 104. The data delivered back to the model 104 may be subjected to technical and content audit, and/or may be used to further train and/or otherwise update the non-user-specific LLM model 104.

Performance of actions for the purpose of reinforcement learning may be manually entered into the system by one or more users, and/or may be automatically determined based on one or more sensors. Subjective emotional outcomes following performance of actions for the purpose of reinforcement learning may be manually entered into the system by one or more users (e.g., as illustrated by the herd reinforcement modules 120 in FIGS. 1A-1B). Physiological responses indicative of psychological and/or neurological responses for the purpose of reinforcement learning may be manually entered into the system by one or more users, and/or may be automatically determined based on one or more sensors.

In some embodiments, feedback information entered by a user may be manually and/or automatically audited with respect to the format and language of the feedback, such that the feedback can be used appropriately uploaded as part of the reinforcement training loop(s) (e.g., as illustrated by technical audit module 122 in FIGS. 1A-1B). A content audit 124 may be applied (manually and/or automatically) to check for prompt content violation with applicable regulations, laws, and/or system policies (e.g., system interoperability policies).

In this way, the personalized system 102 and the non-user-specific system 104 are continually trained and refined, as illustrated by the PHI training update module 126 and HHI herd data update module 128 in FIGS. 1A-1B. As will be explained in greater detail below with respect to at least FIG. 4, the non-user-specific system 104 may further be trained and refined using expert data, as illustrated by HHI expert data update module 130 in FIGS. 1A-1B.

FIG. 2 depicts a method 200 for training, refining, and deploying of a non-user-specific LLM-based happiness and wellbeing support system 204 (referred to in the figures as HHI). The method 200 can correspond to a first training step of an AI language model for assessing and improving happiness and wellbeing of a human user. Optionally, the non-user-specific system 204 described in FIG. 2 may be the same non-user-specific system 104 described in FIGS. 1A-1B.

As shown in FIG. 2, a pre-trained AI language model 202 may be the starting-point for specific configuration of the non-user-specific LLM-based happiness and wellbeing support system 204. In some embodiments, a general-purpose LLM trained on internet communications and internet content may serve as the initial AI language model 202 used in the method 200 of FIG. 2.

The pre-trained AI language model 202 may be subjected to one or more supervised training protocols 205, 206 and/or one or more other refinement protocols based on a filtered training data set comprising one or more of: human philosophy literature and data 208, human psychology literature and data 210, behavioral science literature and data 212, molecular neuroscience literature and data 214, and/or brain imaging literature and data 216. In some examples, the filtered training data set may comprise patents, algorithmic data, study data, definitions, and/or other educational content gathered from the Matter Protocol data 218, described herein. Data in the training data set may be manually and/or algorithmically selected and/or filtered. Data in the training data set may be manually and/or algorithmically weighted for correctness and value. This weighted database of training data may be referred to as a general human wellbeing database 219.

Filtering and/or weighting the training data set for importance and correctness may be performed by human subject-matter experts, by one or more automated analysis algorithms, or both. For example, the training data may be prioritized based on the respective journal impact factors where the scientific paper was published and or the number the selected scientific paper was referenced by others. In addition, approved medical and clinical guidelines may be considered as well as key presentations at the most renown medical and scientific specialty conferences. For literature selection in philosophy and psychology, leading university faculty leaders may be recruited as well as leading university textbooks.

The aggregated training data set may be used to subject the pre-trained AI language model to one or more training protocols, including a training protocol S 206 for scientific correctness and a training protocol R 205 for recommendation accuracy and stability. The training protocol S 206 may be used to refine deterministic and/or probabilistic weighting of data in the training data set (as illustrated by module 220 in FIG. 2). The training protocol S 206 may test the non-user-specific model for scientific correctness based on, e.g., >100,000 term definitions and >1,000 questions addressing yet unresolved controversial topics. The training protocol S 206 may be continuously expanded and refined based on new data.

The training protocol R 205 may be used to refine deterministic and/or probabilistic weighting (as illustrated by module 222 in FIG. 2) and/or to apply one or more updates to the pre-trained AI language model 202 (as illustrated by module 224 in FIG. 2) in order to generate the non-user-specific LLM-based happiness and wellbeing support system model 204. The training protocol R 205, for recommendation accuracy and stability, may test the non-user-specific model 204 for accuracy and stability of general recommendations driving human wellbeing. Training protocol R 205 may be based on >10,000 real-life use cases. Stability assessment may ensure reproducibility of outputs with a range that is still accurate from the scientific impact of a recommendation for a user. Training protocol R 205 may be continuously expanded and refined based on new data.

The generated non-user-specific LLM-based happiness and wellbeing support system model 204 may, in some embodiments, be deployed for use (e.g., for chat-based interaction with users and/or for development of recommendations for actions to improve psychological and/or neurological health). The generated non-user-specific LLM-based happiness and wellbeing support system model 204 may, in some embodiments, serve as a base version of a model for further training and refinement (as explained further herein) to develop a user-specific, personalized LLM-based happiness and wellbeing support system model (e.g., as described with respect to at least FIGS. 1A-1B).

FIG. 3 depicts a method 300 for training, refining, and deploying of a user-specific, personalized LLM-based happiness and wellbeing support system 302 (referred to in the figures as PHI). The method 300 can correspond to a second training step of an AI language model for assessing and improving happiness and wellbeing of a human user. Optionally, the non-user-specific system 304 described in FIG. 3 may be the same non-user-specific system 104 described in FIGS. 1A-1B. The resulting personalized system 302 may be the same personalized system 102 described in FIGS. 1A-1B.

As shown in FIG. 3, non-user-specific LLM-based happiness and wellbeing support system model 304 (e.g., the non-user-specific model 204 described above with reference to FIG. 2) may be the starting-point for user-specific configuration of the personalized LLM-based happiness and wellbeing support system 302.

The non-user-specific model 304 may be subjected to a supervised training protocol 306 and/or one or more other refinement protocols based on a personal training data set 308 comprising one or more of the following for a specific user: clinical physiology data 310, clinical biochemical data 312, full genome analysis data 314, brain scan data (e.g., 7T MRI bran scan data 316), emotional assessment data 318 (e.g., Affect assessment, Flourishing assessment, Life evaluation assessment), and/or memory recording data (e.g., peak memory recording data 320 and/or actual memory recording data 322). The peak memory recording data 320 may include a minimum number of peal memories based on the Matter protocol requirements (e.g., 27 peak memories). The actual memory recording data 322 may include a minimum number of positive memories, from a predetermined past duration of time, as set forth in the Matter protocol (e.g., 50 peak memories). The actual memory recording data 322 may be required to include people involved. Data in the personal training data set may be manually and/or algorithmically selected and/or filtered, as described above with respect to FIG. 2 for the general human wellbeing database 219. Data in the personal training data set may be manually and/or algorithmically weighted for value, as described above with respect to FIG. 2 for the general human wellbeing database 219.

The aggregated personal training data set may be used to subject the non-user-specific AI language model to one or more training protocols 306, including a training protocol S 306 configured to leverage supervised training to apply one or more updates or modifications to the non-user-specific model in order to generate the user-specific LLM-based happiness and wellbeing support system model 302. The training protocols 306 may be performed under supervision of experts and/or the target end-user for whom the system is being configured.

The generated user-specific, personalized LLM-based happiness and wellbeing support system model 302 may, in some embodiments, be deployed for use (e.g., for chat-based interaction with the target user and/or for development of recommendations for actions to improve psychological and/or neurological health of the target user). The generated user-specific LLM-based happiness and wellbeing support system model 302 may, in some embodiments, be further refined by subsequent training, and/or may generate outputs that may be assessed and used for further refinement and/or training of the model itself and/or of other models.

FIG. 4 depicts a method 400 for intermittently and/or continuously updating/refining of a non-user-specific LLM-based happiness and wellbeing support system 402 (referred to in the figures as HHI). The method 400 can correspond to part of the first training step of an AI language model for assessing and improving happiness and wellbeing of a human user. Optionally, the non-user-specific system 404 described in FIG. 4 may be the same non-user-specific system 204 described in FIG. 2 (i.e., the same non-user-specific system 104 described in FIGS. 1A-1B). The resulting updated non-user-specific system 406 may be applied in the processes described with respect to FIGS. 1-2, e.g., in place of the non-user-specific models 104, 204, respectively.

As shown in FIG. 4, an existing version (e.g., as generated based on the method depicted in FIG. 2) of a non-user-specific model 404 may be the starting-point for the update process 400 shown in FIG. 4.

The existing non-user-specific model 404 may be subjected to one or more supervised training protocols 408, 410 and/or one or more other refinement protocols (e.g., as illustrated with respect to modules 412, 413) based on an updated training data set 414 comprising one or more updated data objects as compared to the data set originally used (as described in FIG. 2) to train the existing model 404. The updated data set 414 may include one or more of: new and/or updated human philosophy literature and data, new and/or updated human psychology literature and data, new and/or updated behavioral science literature and data 416, new and/or updated molecular neuroscience literature and data 418, and/or new and/or updated brain imaging literature and data 420. In some examples, the updated training data set 414 includes new and/or updated data from the Matter Protocol 422. As described at least with respect to FIG. 2, data in the updated training data set may be manually and/or algorithmically selected and/or filtered. As described at least with respect to FIG. 2, data in the updated training data set may be manually and/or algorithmically weighted for correctness and value.

The updated training data set 414 may be used to subject the existing non-user-specific model 404 to one or more training protocols 408, 410, including a training protocol S 408 for scientific correctness and a training protocol R 410 for recommendation accuracy and stability. Training protocols S 408 and R 410 may have any one or more characteristics as explained above with respect to at least FIG. 2. For example, the training protocol S 408 may be used to refine deterministic and/or probabilistic weighting of data in the training data set. The training protocol R 410 may be used to refine deterministic and/or probabilistic weighting and/or to apply one or more updates to the existing non-user-specific model in order to generate the updated non-user-specific LLM-based happiness and wellbeing support system model 406.

The updated non-user-specific LLM-based happiness and wellbeing support system model 406 may, in some embodiments, be deployed for use (e.g., for chat-based interaction with users and/or for development of recommendations for actions to improve psychological and/or neurological health). The updated non-user-specific LLM-based happiness and wellbeing support system model 406 may, in some embodiments, serve as a version of a model for further training and refinement (as explained further herein) to develop a user-specific, personalized LLM-based happiness and wellbeing support system model (e.g., personalized model 102 described with respect to FIGS. 1A-1B.

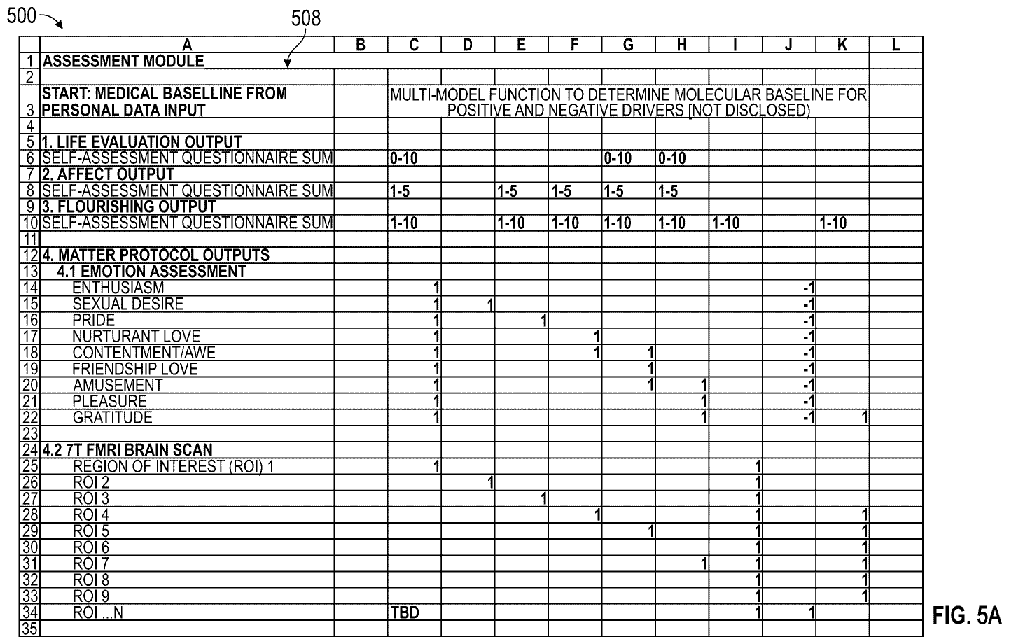

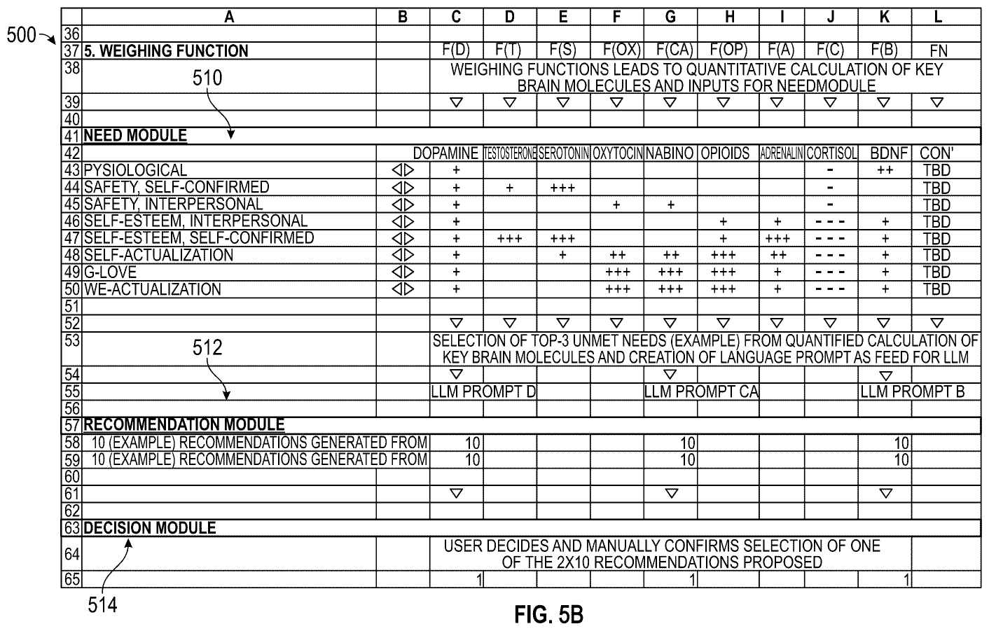

FIGS. 5A-5B shows a table 500 that illustrates an exemplary manner in which an assessment module 508 (e.g., corresponding to assessment module 108 of FIGS. 1A-1B), need module 510 (e.g., corresponding to need module 110 of FIGS. 1A-1B), recommendation module 512 (e.g., corresponding to recommendation module 112 of FIGS. 1A-1B), and decision module 514 (e.g., corresponding to decision module 114 of FIGS. 1A-1B) may work together as part of one or more of the systems and/or methods described herein. In some embodiments, assessment and need modules 508, 510 may be configured to apply one or more algorithms, for example by weighing calculations and matrix calculation to (i) determine critical brain molecules and/or (ii) determine key unmet needs for a particular person. The recommendation module 512 may translate the data into language prompts for one or more language models to process to generate personalized recommendations. The decision module 514 may be configured to allow a user to execute inputs to indicate a user's manual decision as to whether to perform one or more actions recommended by the recommendation module 512.

In one example of using the AI language model for assessing and improving happiness and wellbeing of a human user described herein, the AI language model detects a low Estrogen level (blood biomarker) and an absence of any emotional score on sexual desire in a 30-year-old woman. The user-specific model already has information from the introduction interview that the woman has no siblings, and her mother was very young when she was pregnant with her. The non-user-specific model has knowledge that there could be an unusual early onset of pre-menopause. The recommendation generated by the AI language model would be to see a physician and assess hormone-replacement therapy. Once complete, the user would confirm the task using the AI language model, and the AI language model reinforcement process would begin monitoring the number of memories including sexual desire. Based on a determination that this emotion is increased, the next recommendation generated by the AI language model would be a confirmatory blood test for estrogen.

In another example of using the AI language model for assessing and improving happiness and wellbeing of a human user described herein, the AI language model identifies a link between (a) heavy smartphone use in the evening (behavior biomarker) and a consistently bad sleep (biophysical biomarker: too short deep sleep phase, as detected and acquired from a smart watch or other wearable device) and (b) very low dopamine ratings and very few positive memories given in the Matter mobile application (an emotional biomarker). The AI language would suggest the human stop using phone at least 1 hour before going to sleep. The ultimate decision to change the behavior is however with the user, with a group or team acting on this together, or with the support of a physician or therapist (e.g., if the human has a genetic Cortisol defect that needs to be treated pharmacologically). The execution of this act can be provided to the AI model for reinforcement.

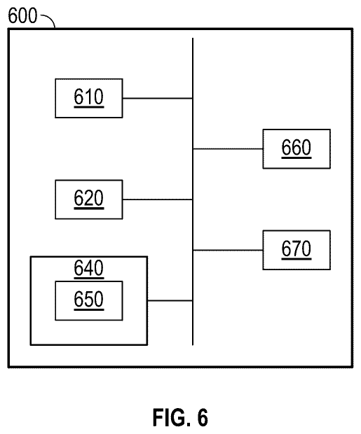

FIG. 6 shows an exemplary computer system 600. System 600 can be any suitable type of processor-based system, such as a personal computer, workstation, server, handheld computing device (portable electronic device) such as a phone or tablet, or dedicated device. System 600 may be configured to execute software causing it to perform all or part of any of the methods described herein. The system 600 can include, for example, one or more of input device 620, output device 630, one or more processors 610, storage 640, and communication device 660. Input device 620 and output device 630 can generally correspond to those described above and can either be connectable or integrated with the computer.

Input device 620 can be any suitable device that provides input, such as a push-button switch, a touch screen, keyboard or keypad, mouse, gesture recognition component of a virtual/augmented reality system, or voice-recognition device. Output device 630 can be or include any suitable device that provides output, such as a display, touch screen, haptics device, virtual/augmented reality display, or speaker.

Storage 640 can be any suitable device that provides storage, such as an electrical, magnetic, or optical memory including a RAM, cache, hard drive, removable storage disk, or other non-transitory computer readable medium. Communication device 660 can include any suitable device capable of transmitting and receiving signals over a network, such as a network interface chip or device. The components of the computing system 600 can be connected in any suitable manner, such as via a physical bus or wirelessly.

Processor(s) 610 can be any suitable processor or combination of processors, including any of, or any combination of, a central processing unit (CPU), graphics processing unit (GPU), field programmable gate array (FPGA), programmable system on chip (PSOC), and application-specific integrated circuit (ASIC). Software 650, which can be stored in storage 640 and executed by one or more processors 610, can include, for example, the programming that embodies the functionality or portions of the functionality of the present disclosure (e.g., as embodied in the devices as described above)

Software 650 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 640, that can contain or store programming for use by or in connection with an instruction execution system, apparatus, or device.

Software 650 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport computer readable medium can include, but is not limited to, an electronic, magnetic, optical, electromagnetic, or infrared wired or wireless propagation medium.

System 600 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

System 600 can implement any operating system suitable for operating on the network. Software 650 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service.

Any one or more embodiments, aspects, claims or other portions of the disclosure provided herein may be combined, in whole or in part, with one another and/or with any other portion of the disclosure provided herein.

### Embodiments

Embodiment 1. A system for training an AI language model for assessing and improving happiness and wellbeing of a human user, the system comprising one or more processors and memory storing instructions configured to be executed by the one or more processors to cause the system to:
receive a pre-trained AI language model;
receive a first training data set comprising non-user-specific training data comprising brain imaging data;
apply one or more supervised training protocols based on the first training data set to modify the pre-trained AI language model to generate a non-user-specific language model configured for happiness and wellbeing support of human users;
receive a second training data set comprising user-specific training data comprising brain-imaging data for a specific user; and
apply one or more supervised training protocols based on the second training data set to modify the non-user-specific language model to generate a user-specific language model configured for happiness and wellbeing support of the specific user.

Embodiment 2. The system of embodiment 1, wherein the instructions are configured to be executed by the one or more processors to cause the system to:
receive input indicating a current happiness and wellbeing state of the specific user;
generate, based on the input indicating the current state, an indication of an unmet happiness or wellbeing need of the user; and
apply the user-specific language model to generate, based on the indication of the unmet need of the user, a first output comprising a first recommendation for an action to induce an improved happiness and wellbeing state of the user.

Embodiment 3. The system of embodiment 2, wherein the instructions are configured to be executed by the one or more processors to cause the system to:
apply the non-user-specific language model to generate, based on the indication of the unmet need of the user, an output comprising a second recommendation for a second action to induce an improved happiness and wellbeing state of the user.

Embodiment 4. The system of embodiment 2, wherein the instructions are configured to be executed by the one or more processors to cause the system to:
receive input indicating an outcome related to the first recommendation; and
apply one or more training updates to the user-specific language model based on the outcome.

Embodiment 5. The system of embodiment 4, wherein the instructions are configured to be executed by the one or more processors to cause the system to:
apply one or more training updates to the non-user-specific language model based on the outcome.

Embodiment 6. A method for training an AI language model for assessing and improving happiness and wellbeing of a human user, the method performed by a system comprising one or more processors and memory, the method comprising:
receiving a pre-trained AI language model;
receiving a first training data set comprising non-user-specific training data comprising brain imaging data;
applying one or more supervised training protocols based on the first training data set to modify the pre-trained AI language model to generate a non-user-specific language model configured for happiness and wellbeing support of human users;
receiving a second training data set comprising user-specific training data comprising brain-imaging data for a specific user; and
applying one or more supervised training protocols based on the second training data set to modify the non-user-specific language model to generate a user-specific language model configured for happiness and wellbeing support of the specific user.

Embodiment 7. The method of embodiment 6, comprising:
receiving input indicating a current happiness and wellbeing state of the specific user;
generating, based on the input indicating the current state, an indication of an unmet happiness or wellbeing need of the user; and
applying the user-specific language model to generate, based on the indication of the unmet need of the user, a first output comprising a first recommendation for an action to induce an improved happiness and wellbeing state of the user.

Embodiment 8. The method of embodiment 7, comprising applying the non-user-specific language model to generate, based on the indication of the unmet need of the user, an output comprising a second recommendation for a second action to induce an improved happiness and wellbeing state of the user.

Embodiment 9. The method of embodiment 7, comprising:
receiving input indicating an outcome related to the first recommendation; and
applying one or more training updates to the user-specific language model based on the outcome.

Embodiment 10. The method of embodiment 9, comprising applying one or more training updates to the non-user-specific language model based on the outcome.

Embodiment 11. A non-transitory computer-readable stage medium storing instructions for training an AI language model for assessing and improving happiness and wellbeing of a human user, the instructions configured to be executed by one or more processors of a system to cause the system to:
receive a pre-trained AI language model;
receive a first training data set comprising non-user-specific training data comprising brain imaging data;
apply one or more supervised training protocols based on the first training data set to modify the pre-trained AI language model to generate a non-user-specific language model configured for happiness and wellbeing support of human users;
receive a second training data set comprising user-specific training data comprising brain-imaging data for a specific user; and
apply one or more supervised training protocols based on the second training data set to modify the non-user-specific language model to generate a user-specific language model configured for happiness and wellbeing support of the specific user.

Embodiment 12. The non-transitory computer readable storage medium of embodiment 11, wherein the instructions are configured to be executed by the one or more processors to cause the system to:
receive input indicating a current happiness and wellbeing state of the specific user;
generate, based on the input indicating the current state, an indication of an unmet happiness or wellbeing need of the user; and
apply the user-specific language model to generate, based on the indication of the unmet need of the user, a first output comprising a first recommendation for an action to induce an improved happiness and wellbeing state of the user.

Embodiment 13. The non-transitory computer readable storage medium of embodiment 12, wherein the instructions are configured to be executed by the one or more processors to cause the system to:
apply the non-user-specific language model to generate, based on the indication of the unmet need of the user, an output comprising a second recommendation for a second action to induce an improved happiness and wellbeing state of the user.

Embodiment 14. The non-transitory computer readable storage medium of embodiment 12, wherein the instructions are configured to be executed by the one or more processors to cause the system to:
receive input indicating an outcome related to the first recommendation; and
apply one or more training updates to the user-specific language model based on the outcome.

Embodiment 15. The non-transitory computer readable storage medium of embodiment 14, wherein the instructions are configured to be executed by the one or more processors to cause the system to:
apply one or more training updates to the non-user-specific language model based on the outcome.

Embodiment 16. A system for using an AI language model to generate a recommendation for an action to induce an improved happiness and wellbeing state of a user, the system comprising one or more processors and memory storing instructions configured to be executed by the one or more processors to cause the system to:
receive input indicating a current happiness and wellbeing state of the specific user;
generate, based on the input indicating the current state, an indication of an unmet happiness or wellbeing need of the user; and
apply a user-specific language model to generate, based on the indication of the unmet need of the user, a first output comprising a first recommendation for an action to induce an improved happiness and wellbeing state of the user, wherein the user-specific language model is trained based brain-imaging data for the user.

Embodiment 17. The system of embodiment 12, wherein the user-specific language model is trained by:
receiving a pre-trained AI language model;
receiving a first training data set comprising non-user-specific training data comprising brain imaging data;
applying one or more supervised training protocols based on the first training data set to modify the pre-trained AI language model to generate a non-user-specific language model configured for happiness and wellbeing support of human users;
receiving a second training data set comprising user-specific training data comprising the brain-imaging data for the user; and
applying one or more supervised training protocols based on the second training data set to modify the non-user-specific language model to generate the user-specific language model.

Embodiment 18. The system of embodiment 17, wherein the instructions are configured to be executed by the one or more processors to cause the system to:
receive input indicating an outcome related to the first recommendation; and
apply one or more training updates to the user-specific language model based on the outcome.

Embodiment 19. A method for using an AI language model to generate a recommendation for an action to induce an improved happiness and wellbeing state of a user, the method performed by a system comprising one or more processors and memory, the method comprising:
receiving input indicating a current happiness and wellbeing state of the specific user;
generating, based on the input indicating the current state, an indication of an unmet happiness or wellbeing need of the user; and
applying a user-specific language model to generate, based on the indication of the unmet need of the user, a first output comprising a first recommendation for an action to induce an improved happiness and wellbeing state of the user, wherein the user-specific language model is trained based brain-imaging data for the user.

Embodiment 20. The method of embodiment 15, wherein the user-specific language model is trained by:
receiving a pre-trained AI language model;
receiving a first training data set comprising non-user-specific training data comprising brain imaging data;
applying one or more supervised training protocols based on the first training data set to modify the pre-trained AI language model to generate a non-user-specific language model configured for happiness and wellbeing support of human users;
receiving a second training data set comprising user-specific training data comprising the brain-imaging data for the user; and
applying one or more supervised training protocols based on the second training data set to modify the non-user-specific language model to generate the user-specific language model.

Embodiment 21. The method of embodiment 20, comprising:
receiving input indicating an outcome related to the first recommendation; and
applying one or more training updates to the user-specific language model based on the outcome.

Embodiment 22. A non-transitory computer-readable storage medium storing instructions for using an AI language model to generate a recommendation for an action to induce an improved happiness and wellbeing state of a user, the instructions configured to be executed by one or more processors of a system to cause the system to:
receive input indicating a current happiness and wellbeing state of the specific user;
generate, based on the input indicating the current state, an indication of an unmet happiness or wellbeing need of the user; and
apply a user-specific language model to generate, based on the indication of the unmet need of the user, a first output comprising a first recommendation for an action to induce an improved happiness and wellbeing state of the user, wherein the user-specific language model is trained based brain-imaging data for the user.

Embodiment 23. The non-transitory computer-readable storage medium of embodiment 22, wherein the user-specific language model is trained by:
receiving a pre-trained AI language model;
receiving a first training data set comprising non-user-specific training data comprising brain imaging data;
applying one or more supervised training protocols based on the first training data set to modify the pre-trained AI language model to generate a non-user-specific language model configured for happiness and wellbeing support of human users;
receiving a second training data set comprising user-specific training data comprising the brain-imaging data for the user; and
applying one or more supervised training protocols based on the second training data set to modify the non-user-specific language model to generate the user-specific language model.

Embodiment 24. The non-transitory computer-readable storage medium of embodiment 23, wherein the instructions are configured to be executed by the one or more processors to cause the system to:
receive input indicating an outcome related to the first recommendation; and
apply one or more training updates to the user-specific language model based on the outcome.

What is claimed is:

1. A system for training an AI language model for assessing and improving happiness and wellbeing of a human user, the system comprising one or more processors and memory storing instructions configured to be executed by the one or more processors to cause the system to:
receive a pre-trained AI language model;
receive a first training data set comprising non-user-specific training data comprising brain imaging data;
apply one or more supervised training protocols based on the first training data set to modify the pre-trained AI language model to generate a non-user-specific language model configured for happiness and wellbeing support of human users;
receive a second training data set comprising user-specific training data comprising brain-imaging data for a specific user; and
apply one or more supervised training protocols based on the second training data set to modify the non-user-specific language model to generate a user-specific language model configured for happiness and wellbeing support of the specific user.

2. The system of claim 1, wherein the instructions are configured to be executed by the one or more processors to cause the system to:
receive input indicating a current happiness and wellbeing state of the specific user;
generate, based on the input indicating the current state, an indication of an unmet happiness or wellbeing need of the user; and
apply the user-specific language model to generate, based on the indication of the unmet need of the user, a first output comprising a first recommendation for an action to induce an improved happiness and wellbeing state of the user.

3. The system of claim 2, wherein the instructions are configured to be executed by the one or more processors to cause the system to:
apply the non-user-specific language model to generate, based on the indication of the unmet need of the user, an output comprising a second recommendation for a second action to induce an improved happiness and wellbeing state of the user.

4. The system of claim 2, wherein the instructions are configured to be executed by the one or more processors to cause the system to:
receive input indicating an outcome related to the first recommendation; and
apply one or more training updates to the user-specific language model based on the outcome.

5. The system of claim 4, wherein the instructions are configured to be executed by the one or more processors to cause the system to:
apply one or more training updates to the non-user-specific language model based on the outcome.

6. A method for training an AI language model for assessing and improving happiness and wellbeing of a human user, the method performed by a system comprising one or more processors and memory, the method comprising:
receiving a pre-trained AI language model;
receiving a first training data set comprising non-user-specific training data comprising brain imaging data;
applying one or more supervised training protocols based on the first training data set to modify the pre-trained AI language model to generate a non-user-specific language model configured for happiness and wellbeing support of human users;
receiving a second training data set comprising user-specific training data comprising brain-imaging data for a specific user; and
applying one or more supervised training protocols based on the second training data set to modify the non-user-specific language model to generate a user-specific language model configured for happiness and wellbeing support of the specific user.

7. The method of claim 6, comprising:
receiving input indicating a current happiness and wellbeing state of the specific user;
generating, based on the input indicating the current state, an indication of an unmet happiness or wellbeing need of the user; and
applying the user-specific language model to generate, based on the indication of the unmet need of the user, a first output comprising a first recommendation for an action to induce an improved happiness and wellbeing state of the user.

8. The method of claim 7, comprising applying the non-user-specific language model to generate, based on the indication of the unmet need of the user, an output comprising a second recommendation for a second action to induce an improved happiness and wellbeing state of the user.

9. The method of claim 7, comprising:
receiving input indicating an outcome related to the first recommendation; and
applying one or more training updates to the user-specific language model based on the outcome.

10. The method of claim 9, comprising applying one or more training updates to the non-user-specific language model based on the outcome.

11. A non-transitory computer-readable stage medium storing instructions for training an AI language model for assessing and improving happiness and wellbeing of a human user, the instructions configured to be executed by one or more processors of a system to cause the system to:
receive a pre-trained AI language model;
receive a first training data set comprising non-user-specific training data comprising brain imaging data;
apply one or more supervised training protocols based on the first training data set to modify the pre-trained AI language model to generate a non-user-specific language model configured for happiness and wellbeing support of human users;
receive a second training data set comprising user-specific training data comprising brain-imaging data for a specific user; and
apply one or more supervised training protocols based on the second training data set to modify the non-user-specific language model to generate a user-specific language model configured for happiness and wellbeing support of the specific user.

12. A system for using an AI language model to generate a recommendation for an action to induce an improved happiness and wellbeing state of a user, the system comprising one or more processors and memory storing instructions configured to be executed by the one or more processors to cause the system to:
receive input indicating a current happiness and wellbeing state of the specific user;
generate, based on the input indicating the current state, an indication of an unmet happiness or wellbeing need of the user; and
apply a user-specific language model to generate, based on the indication of the unmet need of the user, a first output comprising a first recommendation for an action to induce an improved happiness and wellbeing state of the user, wherein the user-specific language model is trained based brain-imaging data for the user.

13. The system of claim 12, wherein the user-specific language model is trained by:
receiving a pre-trained AI language model;
receiving a first training data set comprising non-user-specific training data comprising brain imaging data;
applying one or more supervised training protocols based on the first training data set to modify the pre-trained AI language model to generate a non-user-specific language model configured for happiness and wellbeing support of human users;
receiving a second training data set comprising user-specific training data comprising the brain-imaging data for the user; and
applying one or more supervised training protocols based on the second training data set to modify the non-user-specific language model to generate the user-specific language model.

14. The system of claim 13, wherein the instructions are configured to be executed by the one or more processors to cause the system to:
receive input indicating an outcome related to the first recommendation; and
apply one or more training updates to the user-specific language model based on the outcome.

15. A method for using an AI language model to generate a recommendation for an action to induce an improved happiness and wellbeing state of a user, the method performed by a system comprising one or more processors and memory, the method comprising:
receiving input indicating a current happiness and wellbeing state of the specific user;
generating, based on the input indicating the current state, an indication of an unmet happiness or wellbeing need of the user; and
applying a user-specific language model to generate, based on the indication of the unmet need of the user, a first output comprising a first recommendation for an action to induce an improved happiness and wellbeing state of the user, wherein the user-specific language model is trained based brain-imaging data for the user.

16. The method of claim 15, wherein the user-specific language model is trained by:
receiving a pre-trained AI language model;
receiving a first training data set comprising non-user-specific training data comprising brain imaging data;
applying one or more supervised training protocols based on the first training data set to modify the pre-trained AI language model to generate a non-user-specific language model configured for happiness and wellbeing support of human users;
receiving a second training data set comprising user-specific training data comprising the brain-imaging data for the user; and
applying one or more supervised training protocols based on the second training data set to modify the non-user-specific language model to generate the user-specific language model.

17. The method of claim 16, comprising:
receiving input indicating an outcome related to the first recommendation; and
applying one or more training updates to the user-specific language model based on the outcome.

18. A non-transitory computer-readable storage medium storing instructions for using an AI language model to generate a recommendation for an action to induce an improved happiness and wellbeing state of a user, the instructions configured to be executed by one or more processors of a system to cause the system to:
receive input indicating a current happiness and wellbeing state of the specific user;
generate, based on the input indicating the current state, an indication of an unmet happiness or wellbeing need of the user; and
apply a user-specific language model to generate, based on the indication of the unmet need of the user, a first output comprising a first recommendation for an action to induce an improved happiness and wellbeing state of the user, wherein the user-specific language model is trained based brain-imaging data for the user.
